(19) United States
(12) Patent Application Publication
(10) Pub. No.: US 2024/0280567 A1
(43) Pub. Date: Aug. 22, 2024

(54) PROCESS AND APPARATUS CONFIGURED TO CONDUCT MICROFLUIDIC LOADING AND UNLOADING OF FLUIDS INTO MICROCHANNELS FOR PERFORMING ASSAYS

(71) Applicants: VERSITECH LIMITED, Hong Kong (CN); Advanced Biomedical Instrumentation Centre Limited, Hong Kong (CN)

(72) Inventors: Sammer Ul Hassan, Hong Kong (CN); Ho Cheung Shum, Hong Kong (CN); Pui Chung Lee, Hong Kong (CN)

(73) Assignees: VERSITECH LIMITED, Hong Kong (CN); Advanced Biomedical Instrumentation Centre Limited, Hong Kong (CN)

(21) Appl. No.: 18/437,652

(22) Filed: Feb. 9, 2024

Related U.S. Application Data
(60) Provisional application No. 63/485,743, filed on Feb. 17, 2023.

(51) Int. Cl. G01N 33/543; B01L 3/00

(52) U.S. Cl. CPC G01N 33/54333; B01L 3/502761; B01L 2300/069; B01L 2400/0406

(57) ABSTRACT

A process and an apparatus conduct microfluidic loading and unloading of fluids into microchannels for performing assays, such as immunoassays. The apparatus includes a sequential loading and unloading unit having a movable member dipping a chip having a microchannel including a sample into a cartridge retaining a substance to guide the predetermined substance into the microchannel by capillary action to mix the predetermined substance with the sample. A signal detection unit performs an analysis of the mixed sample and generates an analysis message corresponding to the sample. An output device outputs the analysis message. The process implements operation of the apparatus.

CROSS-REFERENCE TO RELATED PATENT APPLICATIONS

This application claims priority to U.S. Provisional Application No. U.S. 63/485,743, filed Feb. 17, 2023, which is incorporated herein by reference in its entirety.

### FIELD OF THE DISCLOSURE

The present disclosure relates generally to microfluidics technology and molecular biology, and, more particularly, to a process and an apparatus configured to conduct microfluidic loading and unloading of fluids into microchannels for performing assays such as immunoassays.

### BACKGROUND OF THE DISCLOSURE

Laboratory and administrative procedures for patient care can be time-consuming, and diagnosis has been inefficient. Known blood sample analysis systems are pump-based systems, resulting in inhomogeneity or weak spots of pressure. In addition, suction forces can easily develop in the corners of sample retainers or in channels of sample retainers which are further away from other channels, especially in parallel array setups in which the discrepant loading and unloading efficiency would cause large deviations in the incubation time, reaction starting time for enzyme reactions, etc., leading to largely inaccurate results and analysis. Furthermore, a backpressure phenomenon in known pump-based systems causes increasing repelling effects in channels of smaller diameter, which leads to inevitable leakage issues.

In addition, known unloading of channels retaining samples are paper-based or cotton-based with general capillary action in lateral flow assays (LFA), nitrocellulose PVDF, nylon membrane, or agarose gel used in blotting or gel electrophoresis, use a porous wick to transfer molecules through the material, which is inefficient.

Systems are known to perform reactions by mixing fluids. U.S. Pat. No. 8,703,070 B1 describes an immunoassay system using centrifugal force to drive a working fluid to flow into a detection chamber, with capillary action only employed to gradually introduce a magnetic particle diluent into a guiding groove instead of using capillary action to guide a substance into a microchannel to mix with a test sample to be analyzed. U.S. Pat. No. 7,993,534 B2 describes a chemical microreactor having at least one capillary microchannel in a substrate which mixes and delivers a fuel-water mixture from the liquid reservoir instead of guiding a substance into a microchannel to mix with a test sample to be analyzed. US 2021/0140941 A1 describes a biochip having a microchannel provided with a capturing agent for performing cytological analysis by passing a fluid sample containing cells through the microchannel, but lacks using capillary action for guiding a substance into a microchannel to mix with a sample to be analyzed.

### SUMMARY OF THE DISCLOSURE

According to an implementation consistent with the present disclosure, a process and an apparatus are configured to conduct microfluidic loading and unloading of fluids into microchannels for performing assays, including immunoassays. Using the process and the apparatus, consecutive microfluidic loading and unloading into microchannels is conducted to perform enzyme-linked immunosorbent assays.

In an implementation, a process comprises retaining a sample in a microchannel of a chip, retaining a predetermined substance in a well of a cartridge, dipping the chip into the predetermined substance in the well, and guiding the predetermined substance into the microchannel by capillary action, thereby mixing the predetermined substance with the sample. The process further comprises performing an analysis of the mixed sample, generating an analysis message corresponding to the sample, and outputting the analysis message.

Prior to retaining the sample, the process can include coating an interior surface of the microchannel with the sample. After the guiding, the process can incubate the chip with the mixed sample. Alternatively, after the guiding, the process can unload the chip retaining the mixed sample. The unloading can include dipping the chip onto a porous wick or fiber pad. In another alternative implementation, after the guiding, the process can apply a signal generating substance to the mixed sample. The performing of the analysis can include detecting a characteristic of the sample, and the analysis message can include the characteristic. The process can further comprise applying a magnetic field from a magnet to the chip to perform magnetic bead resuspension, and the sample includes a conjugated magnetic bead. Outputting the analysis message can include displaying the analysis message on a display monitor.

In another implementation, a process comprises retaining a sample in a microchannel of a chip, mounting the chip on a movable member configured to move horizontally or vertically, retaining a predetermined substance in a well of a cartridge, positioning the cartridge on a base under the chip, moving the chip downward to dip the chip into the predetermined substance in the well, and guiding the predetermined substance into the microchannel by capillary action, thereby mixing the predetermined substance with the sample. The process further comprises moving the chip upward to remove the chip from the predetermined substance in the well, performing an analysis of the mixed sample, generating an analysis message corresponding to the sample, and outputting the analysis message.

The movable member can be a horizontally movable actuator. Alternatively, the movable member can be a horizontally moving conveyor belt. In another alternative implementation, the movable member can be a vertical moving member. The vertical moving member can be coupled to the horizontally movable conveyor belt. Alternatively, the vertical moving member can be a vertically movable actuator. After the guiding, the process can include incubating the chip with the mixed sample. Performing the analysis can include detecting a characteristic of the sample, and the analysis message can include the characteristic.

In a further implementation, an apparatus comprises a sequential loading and unloading unit, a signal detection unit, and an output device. The sequential loading and unloading unit includes a movable member configured to retain a chip having a microchannel including a sample, and configured to move the chip at least vertically. The sequential loading and unloading unit also includes a base upon which a cartridge is disposed, wherein the cartridge includes a well retaining a predetermined substance. The movable member is configured to move the chip downward to dip the chip into the predetermined substance in the well to guide the predetermined substance into the microchannel by capillary action, thereby mixing the predetermined substance with the sample. The movable member is configured to move the chip upward to remove the chip from the predetermined substance in the well. The signal detection unit is configured, responsive to the chip positioned therein, to perform an analysis of the mixed sample and to generate an analysis message corresponding to the sample. The output device is configured to output the analysis message. The analysis can include a detected characteristic of the sample, and the analysis message includes the detected characteristic. The apparatus can further comprise an incubator configured to incubate the mixed sample.

Any combinations of the various embodiments, implementations, and examples disclosed herein can be used in a further implementation, consistent with the disclosure. These and other aspects and features can be appreciated from the following description of certain implementations presented herein in accordance with the disclosure and the accompanying drawings and claims.

### BRIEF DESCRIPTION OF THE DRAWINGS

FIG. 1 is a schematic of an apparatus, according to an implementation.

FIG. 2 is a schematic of components of the apparatus of FIG. 1 in greater detail.

FIG. 3A is a front view of a sequential loading and unloading apparatus employed by the apparatus of FIGS. 1-2.

FIG. 3B is a side view of the sequential loading and unloading apparatus of FIG. 3A.

FIG. 3C is a top view of the sequential loading and unloading apparatus of FIG. 3A.

FIG. 4 is a flowchart of operation of a process employing the apparatus of FIGS. 1-3C.

FIGS. 5A-5L illustrate an example of the steps of the process shown in FIG. 4.

FIGS. 6A-6N illustrate another example of the steps of the process shown in FIG. 4.

FIGS. 7A-7F illustrate a sequence of loading chips with samples into cartridges.

FIGS. 8A-8F illustrate a sequence of unloading chips with samples.

FIG. 9A illustrates a top side perspective view of a set of chips.

FIG. 9B illustrates a bottom side perspective view of the set of chips of FIG. 9A.

FIG. 10 illustrates a top plan view of the set of chips of FIGS. 9A-9B retaining samples.

FIG. 11 is a graph of absorbance for the samples in the set of chips of FIG. 10.

FIG. 12 is a schematic of a whole-blood microfiltration mechanism.

It is noted that the drawings are illustrative and are not necessarily to scale.

### DETAILED DESCRIPTION OF CERTAIN EMBODIMENTS OF THE DISCLOSURE

Example embodiments and implementations consistent with the teachings included in the present disclosure are directed to a process and an apparatus configured to conduct microfluidic loading and unloading of fluids into microchannels for performing assays, such as immunoassays of biological samples.

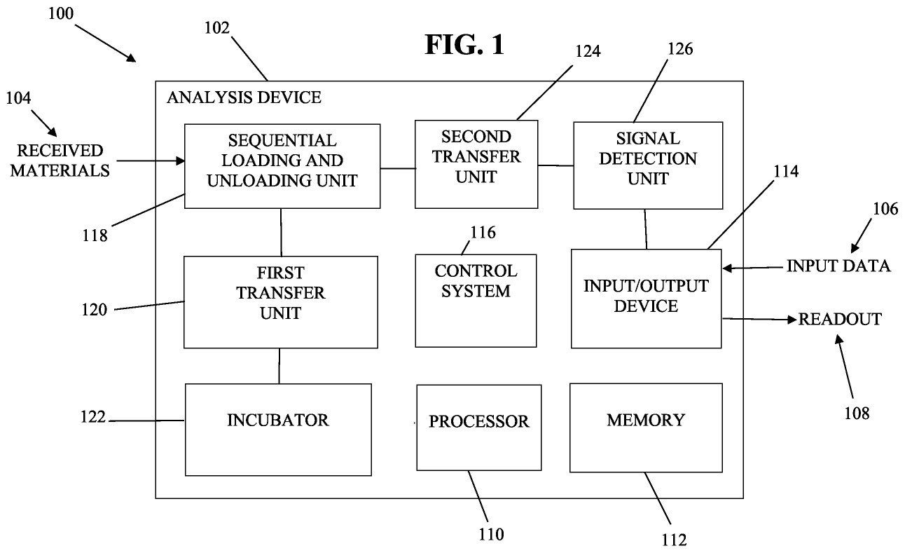

As shown in FIG. 1, in an implementation consistent with the invention, the apparatus 100 is configured to conduct the microfluidic loading and unloading of fluids into microchannels for performing the immunoassays of biological samples. The apparatus 100 includes an analysis device 102 configured to receive materials 104, such as sample of blood or other fluids from a patient, as well as a chip holding the sample and a cartridge holding substances such as fluids in which the sample is dipped to perform assays such as immunoassays, as described in greater detail below. To perform an assay on the blood of the patient, a designated volume of whole blood, such as 40 μL, is first collected from the patient. The blood is then transferred to the inlet of the microchannel component of a chip.

Using such chips dipped in cartridges, an enzyme-linked immunosorbent assay (ELISA) protocol is performed. In one implementation, by dipping the chip into a fluid containing chemicals or biological samples, the microchannels of such chips automatically intake the fluid from the cartridges into the microchannels due to the difference in surface tension or energy between the fluid and microchannel surface. In another implementation, assay components such as antigens and detection antibodies are conjugated on the molecular complex situated on the surface of the microchannel.

In one implementation, the chip has capillary-shaped microfluidic microchannels configured to receive and hold the sample. The microfluidic microchannels of each chip is configured to capture antibodies, aptamers, and other known substances. The microfluidic microchannels of each chip are a major site of reaction and conjugation of reagents. In one implementation, the chip is composed of glass. In another implementation, the chip is composed of plastic.

The chip includes a hollow body having at least one microfluidic microchannel with an opening at the bottom of the chip to receive substances from the cartridge through the capillary pressure in the microchannel. The microchannel of the chip automatically intakes the fluid into the microchannel through the capillary pressure in the microchannel, allowing the sample to be exposed to the received substances. In one implementation, the cartridge is a set of open wells or containers configured and dimensioned to receive at least a portion of the chip dipped into the cartridge containing reagents and other substances to be subsequently loaded into the microchannels of the chip. For example, each microfluidic chip has a size of about 37 mm×16 mm×4 mm, and each cartridge has a size of about 15 cm×12 cm×4 cm.

Referring to FIG. 1, the analysis device 102 is configured to receive the materials 104 as well as input data 106 to perform the assay of the sample and to generate and output the readout 108. In one implementation, the apparatus 100 with the analysis device 102 is automated to perform the assay once the sample in the chip as well as the cartridge of substances are positioned in the device 102. For example, the input data 106 includes a START command input by a user to initiate the automated assaying of the sample. In another example, the input data 106 includes selected commands input by the user to perform different assaying functions. In a further example, the input data 106 includes characters or other known symbols, such as alphanumeric characters or non-alphanumeric symbols. In one implementation, the user enters a name or other identification of a sample being assayed, and so the entered name or identification is associated with the sample.

In one implementation, the analysis device 102 of the apparatus 100 includes a housing configured and dimensioned to have a desktop size, such as 25 cm×15 cm×17 cm. The analysis device 102 includes, in the housing, a processor 110, a memory 112, an input/output device 114, a control system 116, a sequential loading and unloading unit 118, a first transfer unit 120, an incubator 122, a second transfer unit 124, and a signal detection unit 126. For example, the processor 110 is a hardware-based processor such as a microprocessor, and the memory 112 is configured to store instructions and configured to provide the instructions to the hardware-based processor 110. The input/output device 114, the control system 116, the sequential loading and unloading unit 118, the first transfer unit 120, the incubator 122, the second transfer unit 124, and the signal detection unit 126 are configured to implement the instructions provided to the hardware-based processor 110 to perform the assaying process as described below.

In one implementation, the input/output device 114 includes a user interface having at least one button, knob, keyboard, keypad, touchscreen, or any known input device configured to receive the input data 106 as described above. In another implementation, the input/output device 114 includes a user interface having an output device, such as a display monitor, a printer, or any known output device configured to output the readout 108 to the user. For example, the readout 108 includes a message such as a list or table of words or numerical data corresponding to the assayed sample. In another example, the readout 108 includes a graph of data associated with the assayed sample.

In one implementation, the control system 116 is operatively connected to the components 110-114, 118-126 of the analysis device 102. The control system 116 is configured to receive and respond to data and control signals from the processor 110 to control the components 112-114, 118-126 to perform the assaying functions of the analysis device 102. In one implementation, the control system 116 includes a microcontroller, a motor driver, a servo, or other components to control and orchestrate operation of actuators, as described below. In one implementation, the sequential loading and unloading unit 118 includes actuators, arms, and conveyor belts, and the sequential loading and unloading unit 118 is configured to receive at least one chip holding a sample, and receives at least one cartridge having compartments holding substances, such as reagents or other chemicals, into which the at least one chip and sample are loaded or unloaded, as described in greater detail below.

In one implementation, the sequential loading and unloading unit 118 includes a housing with at least one opening, such as a door or seal, through which a user of the analysis device 102, such as a technician, places the chip having a sample and places a cartridge having a substance such as a reagent into retaining areas of the sequential loading and unloading unit 118, as described in greater detail below. In another implementation, the user places the chip having a sample and places the cartridge having a substance such as a reagent into the sequential loading and unloading unit 118 through an opening of the housing of the analysis device 102. In another implementation, the housing of the analysis device 102 has two designated openings, with one opening used to push a cartridge into the sequential loading and unloading unit 118. For example, the opening for inserting the cartridge is a rectangular opening situated in the middle of a front side of the analysis device 102. The other opening in the housing of the analysis device 102 allows a user to insert a chip into the sequential loading and unloading unit 118. The user then attaches the chip directly to a clamp after sliding open a door on the upper half of the front side of the analysis device 102.

The first transfer unit 120 is operatively connected to the sequential loading and unloading unit 118 and the incubator 122. The first transfer unit 120 includes actuators, arms, and conveyor belts, and the first transfer unit 120 is configured, responsive to control signals from the control system 116, to transport the at least one chip holding a sample between the sequential loading and unloading unit 118 and the incubator 122. The incubator 122 includes an interior chamber configured to receive the chips. The incubator 122 includes elements configured to be responsive to control signals from the control system 116, such as heating elements, cooling elements, and humidity controlling elements, to control the condition of the samples during the assaying of the samples. In one implementation, the incubator 122 is sealed and equipped with 12V 7 W polyimide heater films having the size of 25 mm×50 mm, which are able to produce the desired temperature from 30° C. to 80° C., fulfilling the requirements of most immunoassays.

The second transfer unit 124 is operatively connected to the sequential loading and unloading unit 118 and the signal detection unit 126. In one implementation, the second transfer unit 124 includes actuators, arms, and conveyor belts, and the second transfer unit 120 is configured, responsive to control signals from the control system 116, to transport at least one chip holding a sample between the sequential loading and unloading unit 118 and the signal detection unit 126.

The signal detection unit 126 is operatively connected to input/output device 114. The signal detection unit 126 is configured to receive an assayed sample from the sequential loading and unloading unit 118, and responsive to control signals from the control system 116, to generate and output a signal corresponding to and representing a detected characteristic of the assayed sample. The signal detection unit 126 outputs the characteristic detection signal to the input/output device 114. In response to the characteristic detection signal, the input/output device 114 generates and outputs the readout 108 corresponding to the assayed sample, as described above.

In one implementation, characteristic detection signals are collected from multiple microchannels in the chips retaining the samples. Depending on the assay, colorimetric or fluorescence detection is carried out with light or laser induced spectroscopy, and subsequent data collection and analysis is performed with a photodiode on the absorbance of the sample. The input/output device 114 is configured to output the analysis of the samples. For example, a standard curve is constructed with the standard antigen levels produced with serial dilution, and a desired patient biomarker or antigen level is extrapolated and calculated using the curve constructed curve.

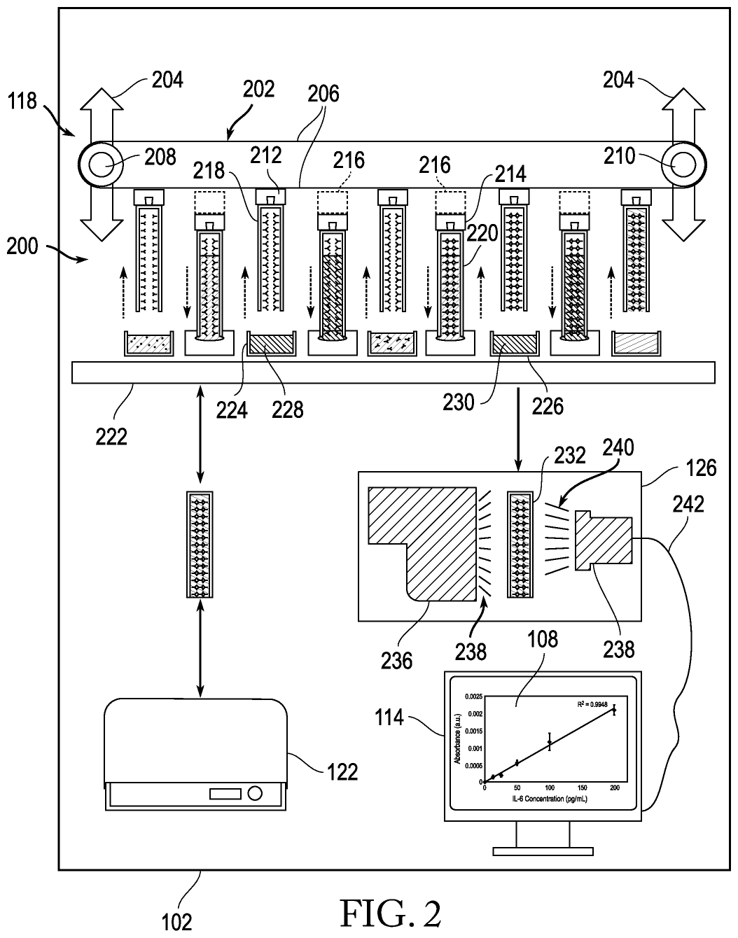

FIG. 2 is a schematic of components of the apparatus 100 in greater detail. In one implementation, the sequential loading and unloading unit 118 includes a transport mechanism 200 having a horizontal movement mechanism 202 and a vertical movement mechanism 204. In one implementation, the horizontal movement mechanism 202 includes a conveyor belt 206 with portions horizontally moving by rotation of wheels 208, 210. In one implementation, the vertical movement mechanism 204 includes a linear actuator. In another implementation, the vertical movement mechanism 204 includes a vertical railway.

At least one clamp 212, 214 is operatively coupled to a portion of the conveyor belt 206. For example, vertical movement device 216, such as a linear actuator, operatively couples the clamp 212, 214 to the portion of the conveyor belt 206. The vertical movement device 216 moves a respective clamp 212, 214 vertically away from or towards the conveyor belt 206. Each clamp 212, 214 is configured to releasably retain a respective chip 218, 220 holding a respective sample. A user places the chips 218, 220 with the samples onto respective clamps 212, 214. Each clamp 212, 214 and a respective chip 218, 220 is moved in a vertical direction by the vertical movement device 204, with the vertical direction perpendicular to the horizontally oriented conveyor belt 206.

The sequential loading and unloading unit 118 includes a base 222 within the housing of the sequential loading and unloading unit 118. At least one cartridge 224, 226 is disposed on the base, with each cartridge 224, 226 retaining a substance 228, 230, respectively. In one implementation, the base 222 includes a clamp or other retaining mechanism configured to releasably retain each cartridge 224, 226 on the base 222. Accordingly, during the process of dipping chips 218, 220 into the cartridges 224, 226, the retaining mechanism on the base 222 prevents the cartridges 224, 226 from moving to ensure that the chips 218, 220 and the cartridges 224, 226 are aligned for dipping.

The vertical movement mechanism 204 is configured to vertically move the entire horizontal movement mechanism 202 as well as all of the clamps 212, 214 and chips 218, 220 upward away from or downwards towards the base 222, the cartridges 224, 226, and the substances 228, 230. Using control signals, the control system 116 controls the vertical movement mechanism 204 to vertically move the entire horizontal movement mechanism 202. Accordingly, by moving the entire horizontal movement mechanism 202 upward, the sequential loading and unloading unit 118 is configured to reset the position of all of the chips 218, 220 to be in a loading position. In the loading position, a user is allowed to place or remove all or individual chips 218, 220 onto the horizontal movement mechanism 202.

Each vertical movement device 216 is configured to vertically and selectively move individual clamps 212, 214 and individual chips 218, 220 thereon, respectively, upward away from or downwards towards the base 222, the cartridges 224, 226, and the substances 228, 230. Using control signals, the control system 116 controls the vertical movement devices 216 to vertically and selectively move individual clamps 212, 214 and individual chips 218, 220 thereon, to selectively control dipping of individual chips 218, 220 into respective cartridges 224, 226 having respective substances 228, 230.

In one implementation, the user places the cartridges 224, 226 and the respective substances 228, 230 onto the base 222 through the opening of a housing of the sequential loading and unloading unit 118. Referring to the cartridges 224, 226, the respective substances 228, 230 include, for example, an antigen into which the chip 218, 220 and the sample therein is dipped. The vertical movement device 216 moves each chip 218, 220 into at least one of the cartridges 224, 226 to expose the samples to the substances 228, 230 in the cartridges 224, 226.

As shown in FIG. 2, the clamps 212, 214 and the chips 218, 220, respectively, are spaced in predetermined positions on the conveyor belt 206, and the cartridges 224, 226 are spaced in predetermined positions on the base 222, with the predetermined positions of the cartridges 224, 226 being complementary and matching the predetermined positions of the chips 218, 220 to ensure proper and accurate dipping of the chips 218, 220 into the substances 228, 230 in the cartridges 224, 226, respectively.

In one implementation, the sequential loading and unloading unit 118 is situated in an upper portion of the analysis device 102, such that the chips 218, 220 are engaged by the respective clamps 212, 214 on the sliding conveyor belt 206 to horizontally transport the chips 218, 220. In one implementation, the belt system of the conveyor belt 206 is hinged on a vertical railway, as the vertical movement mechanism 204, enabling the dipping of the chips 218, 220 into the substances 228, 230 to be performed wholly inside the analysis device 102. In one implementation, using the vertical movement devices 216, the clamps 212, 214 move vertical to the conveyor belt 206 by a dipping distance controlled by the vertical movement devices 216. The clamps 212, 214 move horizontally by a horizontal traveling distance controlled by the horizontal movement mechanism 202. In one implementation, the dipping distance and the horizontal traveling distance are manually set at a factory. In another implementation, the dipping distance and the horizontal traveling distance are calibrated with sensors.

In a further implementation, the dipping distance and the horizontal traveling distance are preprogrammed by a 2.5 dimensional computer aided design (CAD) system. The dipping distance and the horizontal traveling distance are computed and executed through an Arduino processor located in the analysis device 102. In another interpretation, the width and distance from well to well of the cartridges are standardized in the range of products provided, and so users only need to input the number of microchannels required, order of dipping, and duration required for each loading, for the system to perform the automated dipping and unloading protocol. For example, a user inputs the number of microchannels required, order of dipping, and duration required for each loading using the input/output device 114. In an alternative implementation, users customize and save certain orders and durations for the assay processes as a default or saved profile in which users easily re-use a profile for a repeated assay.

As shown in FIG. 2, at least one of the chips 218, 220 are transported from the sequential loading and unloading unit 118 to the incubator 122, and later transported from the incubator 122 back to the sequential loading and unloading unit 118. For example, as described above, the first transfer unit 120 performs such transporting of the chips 218, 220 transported to the incubator 122, and transporting of the chips 218, 220 transported from the incubator 122 back to the sequential loading and unloading unit 118. When the various assaying steps are performed by the sequential loading and unloading unit 118 and the incubator 122, the processed chips 218, 220 with the assayed samples are transported from the sequential loading and unloading unit 118 to the signal detection unit 126.

Once a chip with an assayed sample, such as the chip 232 and the sample 234 shown in FIG. 2, is positioned within the signal detection unit 126, the signal detection unit 126 detects the characteristics of the sample. In one implementation, the signal detection unit 126 includes a transmitter 236 and a receiver 238. The transmitter 236 is configured to transmit a transmitted emission 238 to the chip 232 and the sample 234. For example, the transmitted emission 238 is an electromagnetic wave. In another example, the transmitted emission 238 is a sound wave. Upon receiving the transmitted emission 238, the sample 234 responds to the transmitted emission 238 by generating and outputting a characteristic emission 240. The characteristic emission 240 represents characteristics of the sample 234. The receiver 238 receives the characteristic emission 240, and generates and outputs a detection signal corresponding to or representing the characteristics of the sample 234. The receiver 238 is operatively connected to the input/output device 114. For example, a wired connection 242 electronically connects the receiver 238 to the input/output device 114 to convey the detection signal. In another example, the receiver 238 is operatively connected to the input/output device 114 by a wireless connection. The input/output device 114 then generates and outputs the readout 108, as described above.

Optionally, after detection of the characteristics of the sample by the signal detection unit 126, the assayed samples are transported from the signal detection unit 126 back to the sequential loading and unloading unit 118 for removal by a user, such as a technician.

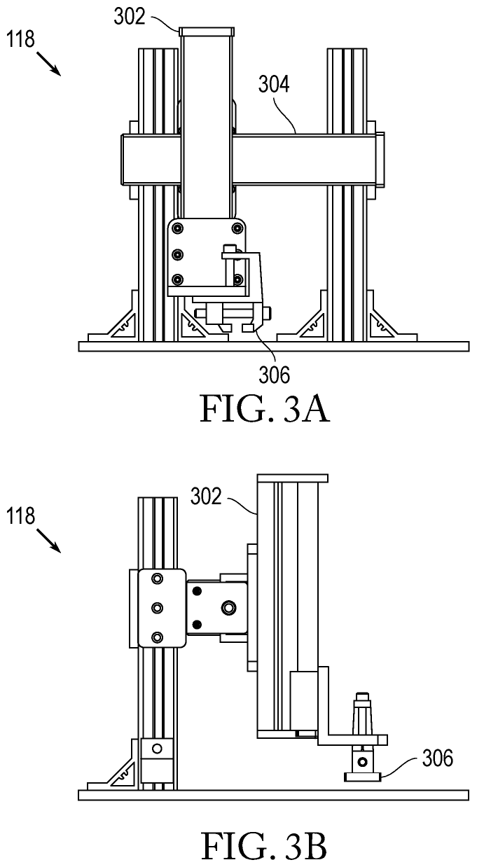

In an alternative implementation, the sequential loading and unloading unit 118 as shown in FIGS. 3A-3C is a sophisticated mechanical device equipped with two high-precision linear actuators 302, 304, enabling precise and dynamic movements of a microfluidic chip along both vertical and horizontal axes. The first linear actuator 302, positioned vertically, includes a robust lead screw mechanism with a pitch of 2 mm per revolution. The actuator 302 is responsible for the vertical displacement of a chip held by a clamp 306 in the Z-direction, precisely controlled within a range of ±10 mm, ensuring accurate positioning during experiments and operations. The second linear actuator 304, oriented horizontally, employs a screw drive mechanism with a pitch of 1 mm per revolution. The second linear actuator 304 facilitates smooth horizontal movement of a chip, enabling displacement in an X-Y plane within a range of ±20 mm. In one implementation, the linear actuators 302, 304 are equipped with high-resolution encoders, providing feedback for closed-loop control and positional accuracy down to micrometer-scale.

The sequential loading and unloading unit 118 is operatively coupled to the control system 116 shown in FIG. 1. In one implementation, the control system 116 is an integrated control system, including microcontrollers and motor drivers. The control system 116 is configured to orchestrate synchronized operation of the actuators 302, 304, ensuring coordinated and precise positioning of the microfluidic chip. The advanced mechanical setup of the sequential loading and unloading unit 118 operated by the control system 116 enables users to manipulate a selected chip with exceptional accuracy, facilitating intricate fluidic experiments and analysis in the realm of microscale technologies.

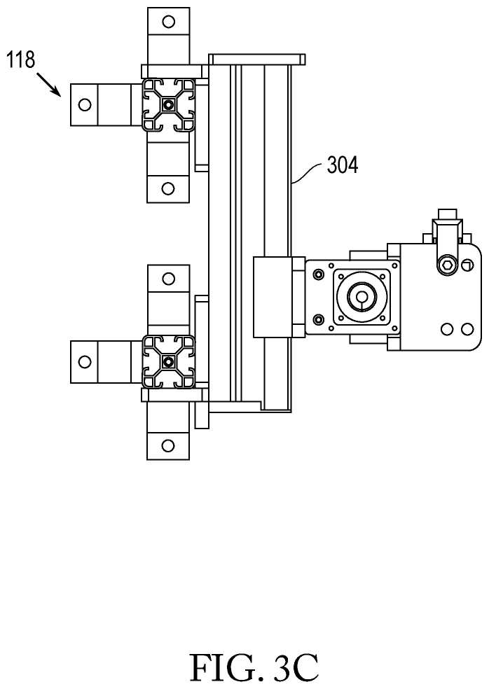

As shown in FIG. 4 in conjunction with FIGS. 1-2, the apparatus 100 including the analysis device 102 performs the process 400 including the step of receiving materials including a sample, a chip 218, and a cartridge 224 holding a substance 228 in step 402. The process 400 then performs securing a sample in the chip 218 in step 404. The sample is inserted into the microchannel in the hollow body of the chip 218. For example, the sample is coated on an interior surface of the microchannel. Optionally, a cap is secured to an end of the chip 218 to retain the sample in the chip 218. The process 400 then positions the cartridge 224 and the chip 218 including the sample in the analysis device 102 in step 406. As described above, a user such as a technician inserts the chip 218 and the cartridge 224 into an opening in the housing of the sequential loading and unloading unit 118 to position the cartridge 224 and the chip 218 including the sample in the sequential loading and unloading unit 118 of the analysis device 102.

The process 400 then sequentially moves the chip 218 into and out of the cartridge 224 and into and out of the incubator 122 in step 408 to perform assaying steps on the sample. For example, the chip 218 includes a hollow body including a microchannel having at least one opening at the bottom of the chip 218 to receive substances from the cartridge 224 through the capillary pressure or action in the microchannel. The microchannel of the chip 218 automatically intakes the substance 228 into the microchannel through the capillary pressure or capillary action in the microchannel, allowing the sample to be exposed to and mixed with the received substances. For example, in step 408, the chip 218 is transported between the sequential loading and unloading unit 118 and the incubator 122 using the first transfer unit 120. The process 400 then conjugates a signal generating substance into the assayed or processed sample in step 410. In one implementation, one of the cartridges in the sequential loading and unloading unit 118 retains the signal generating substance, and the chip 218 with the sample is dipped into the signal generating substance to perform step 410. In another implementation, the chip 218 with the processed sample is transported from the sequential loading and unloading unit 118 to the signal detection unit 126, and a mechanism of the signal detection unit 126 inserts the signal generating substance into the chip 218 with the sample to perform step 410.

In one implementation, the signal generating substance is a signal generating reagent, such as chromogenic substrate TMB or ABTS for enzyme-linked secondary antibodies and aptamers, or a fluorophore-conjugated detection antibody or aptamer with fluorophores such as FITC or Amplex Red. Once the final signal-generating reagent has been loaded into the chip with the sample, the second transfer unit 124 transport the chip from the sequential loading and unloading unit 118 to the signal detection unit 126. In one implementation, the chip with the sample to be analyzed is placed in a sealed and transparent plate for the collection of colorimetric and fluorescent signals.

The process 400 then performs analysis on the processed sample in step 412 using the signal detection unit 126. For example, in step 412, the chip 218 with the processed sample is transported from the sequential loading and unloading unit 118 by the second transfer unit 124. In one implementation, the analysis of the processed sample in step 412 includes generating and outputting a characteristic signal corresponding to and representing the characteristics of the sample.

To perform colorimetric or fluorescence detection, the signal detection unit 126 uses light or laser-induced spectroscopy, and subsequent data collection and analysis is performed with a photodiode on the absorbance of the sample. The signal detection is carried out by a scanning probe, which holds an excitatory LED or laser source. In one implementation, the sealed and transparent plate is affixed on an electric linear actuator, such that the microchannels of the chip with the samples move horizontally through the light source one by one. After being excited by the light source, fluorophores emit a specific light wave in the designated spectrum, such as light having a wavelength of 550 nm. The signal detection unit 126 includes photoelectric sensors located across the light source, and the photoelectric sensors collect and measure the emittance spectrum of the microchannels. The transmitted light collected from a sample during colorimetric detection are converted into absorbance measurements.

The process 400 then generates and outputs an analysis message, such as the readout 108, in step 414 using the input/output device 114. In one implementation, the input/output device 114 includes image processing software. After calibrating and analyzing the emittance spectrum in the image processing software, a standard curve is constructed with the standard antigen levels produced with serial dilution, and the desired patient biomarker or antigen level is extrapolated and calculated using the curve.

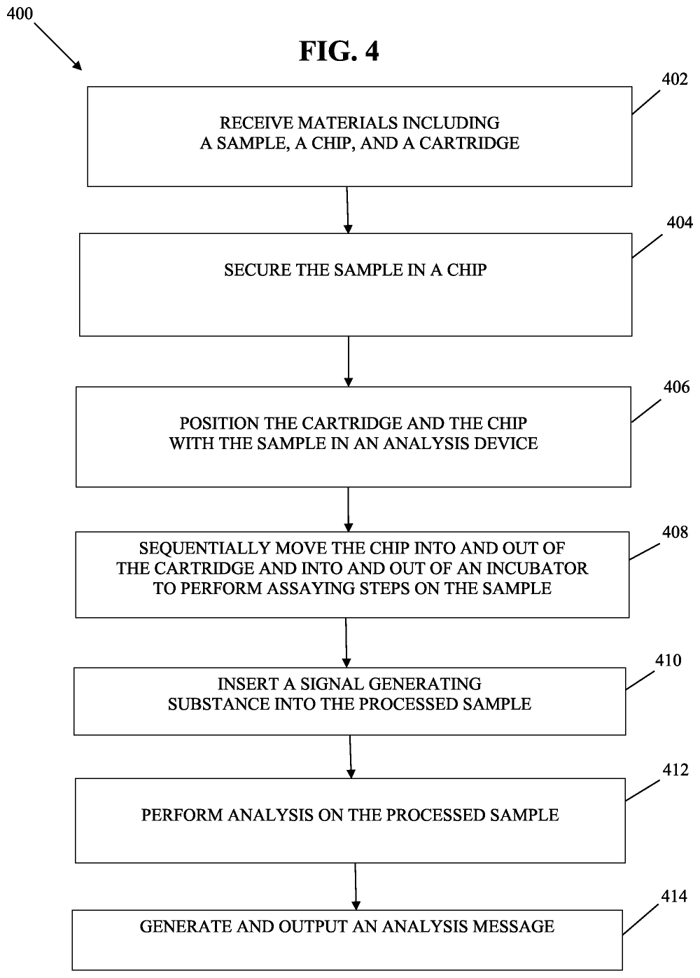

FIGS. 5A-5L illustrate an example of the steps of the process 400 shown in FIG. 4. In FIG. 5A, an internal microchannel of a chip 500 is coated with a sample 502, such as primary antibody (Ab). In FIG. 5B, a cartridge 504 includes a substance, such as an antigen 506. In FIG. 5C, the chip 500 with the sample 502 is loaded into and then removed from the antigen 506. The microchannel of the chip 500 automatically intakes the fluid into the microchannel through the capillary pressure in the microchannel. In FIG. 5D, the chip 500 is transported to the incubator 122 for incubation. In FIG. 5E, the incubated chip is unloaded by dipping the chip 500 onto an external capillaric element 508, such as a porous wick or fiber pad, and the chip 500 is transported from the incubator 122. In FIG. 5F, the chip 500 is loaded into a washing buffer 510. In FIG. 5G, the chip 500 is unloaded from the washing buffer 510. Optionally, FIGS. 5H-5L are performed, depending on the experiment. In FIG. 5H, the chip 500 is loaded into and then unloaded from a different cartridge 512 retaining a different substance 514. In FIG. 5I, the chip 500 is transported to the incubator 122 for incubation. In FIG. 5J, the chip 500 is unloaded and transferred from the incubator 122. In FIG. 5K, another wash of the chip 500 is performed using a washing buffer, and a cartridge 516 includes a substrate providing a signal generating substance. The substrate is applied to the processed sample 518 in the chip 500. In FIG. 5L, the processed sample 518 and the substrate providing the signal generating substance are analyzed by the signal detection unit 126.

Referring to FIGS. 5A-5L, in one implementation, standard ELISA procedures are performed through a dipping mechanism of the chip. As shown in FIG. 5A, the inner surface of microchannels in the chip 500 are precoated with a primary or detection antibody. By dipping the chip on fluid containing chemicals or biological samples, the microchannel would automatically intake the fluid into the microchannels through the capillary pressure in the microchannels. The assay commences with dipping the chip into antigen-containing fluids 506, such as blood or saliva eluded in medium, as shown in FIG. 5C. The microchannel intakes the antigen which binds the receptors on the immobilized primary antibody in the microchannel. The antigen is incubated briefly inside the microchannel in FIG. 5D, and is then unloaded from the chip 500 as shown in FIG. 5E by dipping the chip 500 onto an external capillaric clement 508, such as a porous wick or fiber pad. The microchannel is then washed by washing buffer shown in FIG. 5F to remove any non-specific binding of antigen by dipping on washing buffer and fiber pad sequentially. A secondary antibody linked with enzyme or fluorophore is then loaded into the chip 500 as shown in FIG. 5H using the same dipping mechanism, and is bound on the antibody-antigen conjugate, followed by another round of washing in FIG. 5K. Colorimetric or fluorescence analysis can then be performed in FIG. 5L with or without an addition of substrate such as HRP, depending on the antibody design.

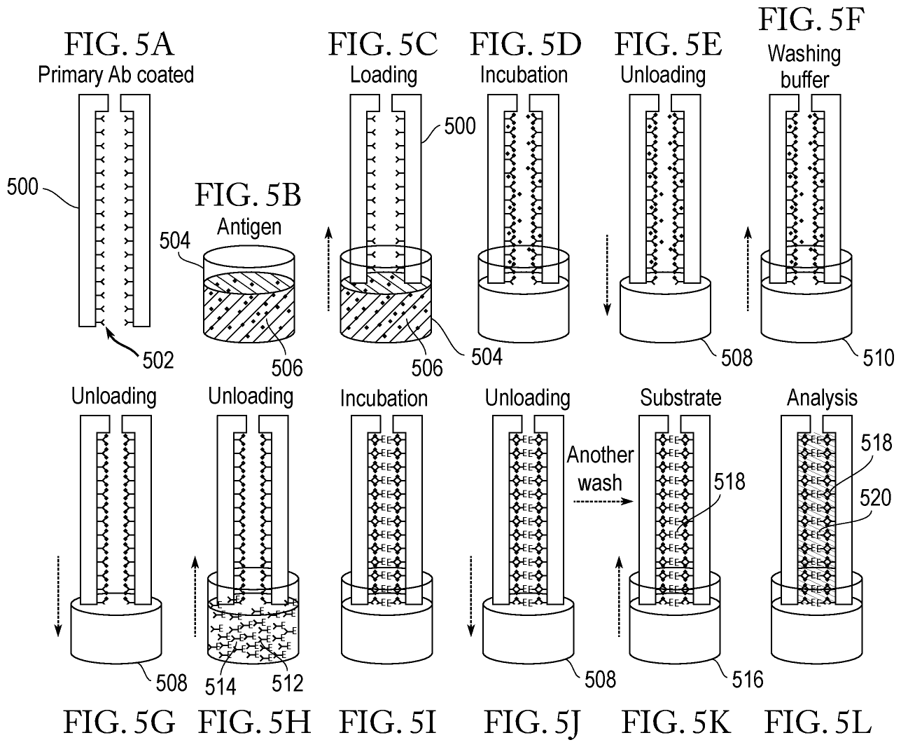

FIGS. 6A-6N illustrate another example of the steps of the process shown in FIG. 4, performed in conjunction with magnetic beads, which is another technique used in microfluidics. In FIG. 6A, a chip 600 has an uncoated microchannel 602. In FIG. 6B, a cartridge 604 retains antibody conjugated magnetic beads 606. In FIG. 6C, a magnetic field 612 is applied by a magnet 614 to perform magnetic bead fixation, where magnetic beads are held within the microchannel 602 through external magnetic attraction. In FIG. 6D, unloading of the fluid while retaining the magnetic beads 606 inside the microchannel 602 is performed by dipping the chip 600 onto an external capillaric clement 608, such as a porous wick or fiber pad. In FIG. 6E, the chip 600 is dipped into an antigen 610. In FIG. 6F, the magnetic field 612 is removed to perform magnetic bead resuspension to enhance interaction between antigen 610 and magnetic beads 606. In FIG. 6G, the magnetic field 612 is reapplied by magnet 614 to perform magnetic bead fixation while unloading is performed. In FIG. 6H, a washing buffer is applied. In FIG. 6I, magnetic bead resuspension is performed again by removing the magnetic field 612 from a magnet 614. In FIG. 6J, the magnetic field 612 is reapplied by magnet 614 to perform magnetic bead fixation while unloading is performed. In FIG. 6K, an antibody-enzyme 616 is applied to the chip having the magnetic beads 606. In FIG. 6L, washing is performed. In FIG. 6M, a substrate 620 including a signal generating substance is applied to the chip 600 having the magnetic beads 606. In FIG. 6N, the substrate 620 providing the signal generating substance are analyzed by the signal detection unit 126.

Referring again to FIGS. 6A-6N, in one implementation, a primary or detection antibody is first conjugated with magnetic beads 606 which is suspended in a medium, as shown in FIG.

6B. The beads 606 can be fixated or resuspended inside the microchannel in FIG. 6C by applying and removing an external magnet, such that fixation is used to hold the desired reaction mixture inside the microchannel 602 to proceed to subsequent steps, and resuspension is used to enhance reaction speed inside the microchannel 602 for conjugation steps and washing. The assay begins with dipping an uncoated chip 600 in FIG. 6A into the magnetic beads 606 in FIG. 6C, attaching the external magnet, followed by unloading of the fluid through dipping on a fiber pad as shown in FIG. 6D. Then, antigen-containing fluids, such as blood or saliva eluded in medium, are loaded into the microchannel in FIG. 6E, followed by resuspension of the magnetic beads in FIG. 6F. The antigen then binds to the receptors on the freely moving antibody-bead conjugates inside the microchannel 602. The beads are then fixated, and residual fluids are unloaded from the chip in FIG. 6G by dipping on a fiber pad 608. The microchannel 602 is then washed in FIG. 6H by loading washing buffer and resuspending the beads in FIG. 6I to remove any non-specific binding of antigen. The residual fluid is then unloaded after fixating the beads in FIG. 6J. Following such a sequence of steps, a secondary antibody linked with enzyme or fluorophore as the sample 616 is then loaded into the chip in FIG. 6K, and is bound on the antibody-antigen conjugate, followed by another round of washing in FIG. 6L. Colorimetric or fluorescence analysis is performed in FIG. 6N with or without an addition of substrate such as HRP in FIG. 6M, depending on the antibody design.

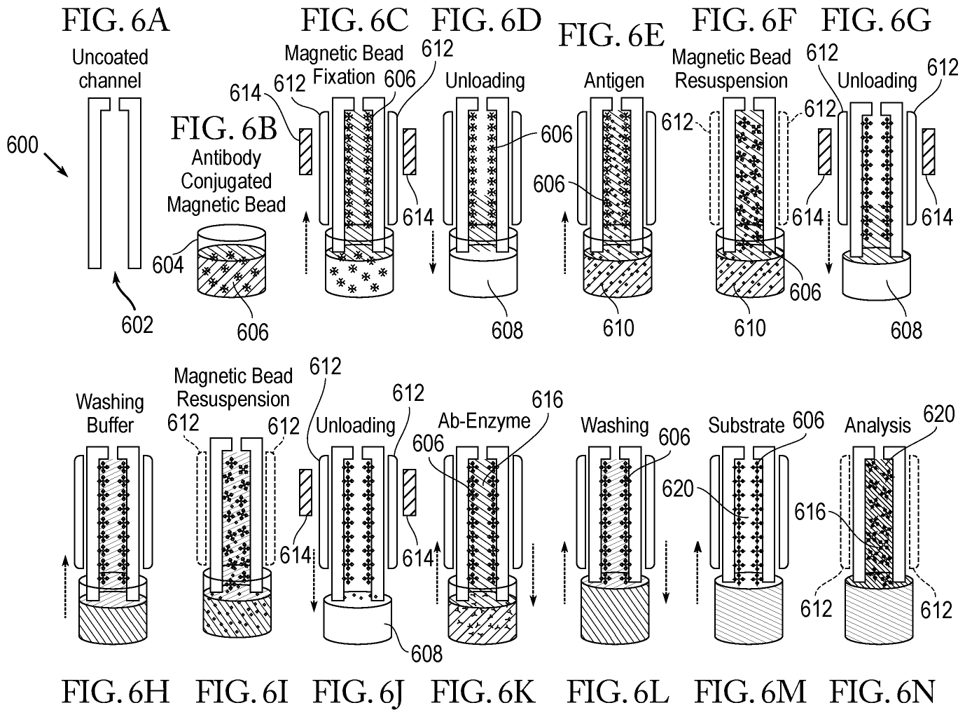

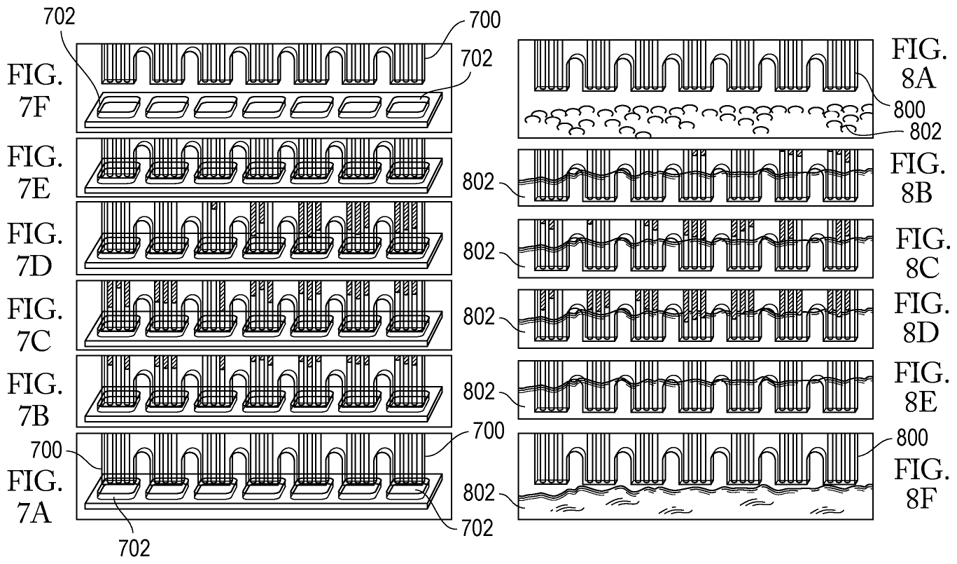

FIGS. 7A-7F illustrate a sequence of loading chips 700 with samples into cartridges 702 with substances, with each set of chips in the sequence imaged at 500 ms intervals. Fluid in contact with the opening of the microchannels of the chips 700 rapidly flows into the microchannels, allowing chemical and biological processes to occur. FIGS. 8A-8F illustrate a sequence of unloading chips 800 with samples, with each set of chips in the sequence imaged at 500 ms intervals. The unloading of the chips 800 is performed by dipping the chips 800 onto an external capillaric element 802, such as a porous wick or fiber pad. Such unloading in FIGS. 8A-8F is performed using capillary pressure. The unloading of the fluid is also rapid upon contact with the absorbing fiber pad that is higher in capillary pressure compared to the microchannel. Flow velocity analysis has been conducted and the highest average velocity for loading is about 2.55 mm/s, while the average loading velocity is about 1.54 mm/s. Alternatively, the average velocity for unloading is about −2.8 mm/s, while the highest average unloading velocity is about −6 mm/s when approaching the opening of the microchannel.

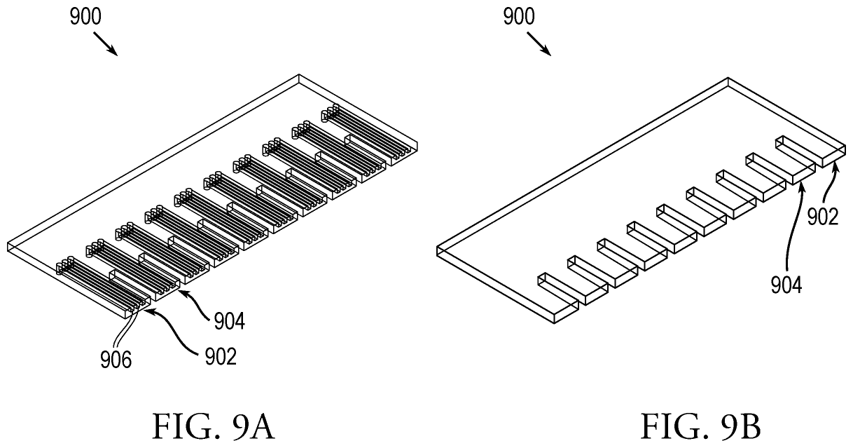

As shown in FIGS. 9A-9B, in one implementation, a set 900 of chips 902, 904. Each chip 902, 904 includes at least one microchannel 906. For example, the set 900 includes a top layer and a bottom layer. As shown in FIG. 9A, the top layer is formed by an extrusion process to be configured with 30 microchannels and with each microchannel being 500 microns (μm) wide and 500 microns (μm) deep. The length of each microchannel is 1 centimeter (cm), starting from the bottom side of the top layer along the vertical axis of the chip. Circular openings of 800 microns in diameter above each of the extruded microchannels protrude the entirety of the top layer of the chip, forming a chimney-like configuration for each of the microchannels. In one implementation, three microchannels 906 are grouped in a chip 902, 904, and each of the three microchannels is separated by a trough which is 2 millimeters (mm) wide. As shown in FIG. 9B, the bottom layer is a thin film with no extrusions formed therein. When the top layer is covered and sealed by the bottom layer, a working fluidic microchannel system with fluidic inlets on the bottom of the chip is formed, and pressure outlets through the circular opening at the top of the microchannels is also formed.

In one implementation, the top and bottom layers of the set 900 of chips 902, 904 are fabricated through the following steps: creating a prototype of microchip by using AutoCAD software; converting the prototype of AutoCAD design into toolpaths for machining the microchip; using the toolpaths to mill microchannels or drill holes at designated positions within the microchip to create inlets and outlets; employing the toolpaths to mill holes and connecting microchannels within the microchip, ensuring precision and consistency; and cutting out a product of microchip based on the design using the toolpaths. The top layer is compose of polymethyl methacrylate (PMMA), and the bottom layer is composed of polymethyl methacrylate (PMMA) or a biocompatible adhesive thin film. In an alternative implementation, the dimensions of the set 900 of chips 902, 904, the specific number of microchannels, the width and depth of microchannels, along with other specifications of the chip can be modified depending on the needs and variations of the application to perform assaying of samples.

In one implementation, the inner surfaces of the extruded microchannels of the

microfluidic chips are treated prior the subsequent assays to be performed with the chip. For example, a proteophillic agent, A20, is loaded into the chip and incubated at room temperature for 20 seconds, which is then aspirated out of the chip. The chip is then baked at 65° C. for 10 minutes for the self-assembly of a proteophillic thin film on the surface for better protein immobilization performance of the chip in the later stages of the assay. A captured antibody or aptamer diluted in a suitable buffer, such as PBS or BSA, is then loaded into the chip for overnight incubation in 4° C. for immobilization before the commencement of the assay.

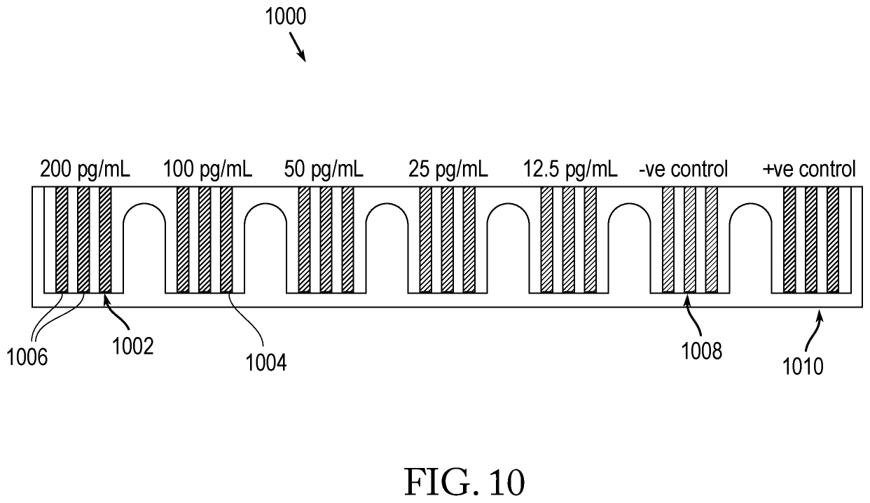

FIG. 10 illustrates a top plan view of a set 1000 of chips 1002, 1004, with each chip 1002, 1004 including at least one microchannel 1006 retaining samples having concentrations of 200 pg/mL, 100 pg/mL, 50 pg/mL, 25 pg/mL, and 12.5 pg/mL. The set 1000 also includes a pair of control chips 1008, 1010.

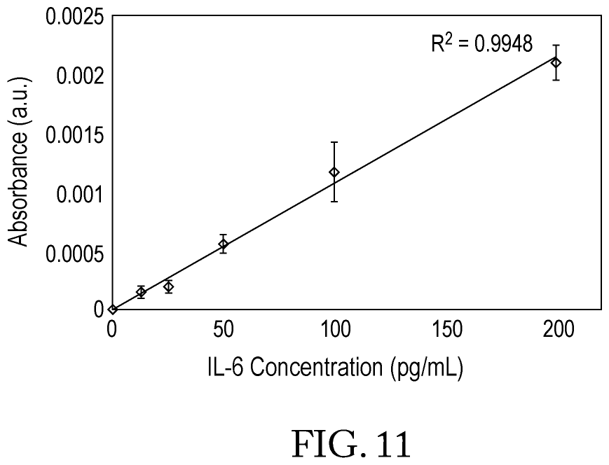

FIG. 11 is a graph of absorbance for the samples in the set 1000 of chips of FIG. 10, with data points corresponding to concentrations of 200 pg/mL, 100 pg/mL, 50 pg/mL, 25 pg/mL, and 12.5 pg/mL. In one implementation, using the signal detection unit 126, a standard curve of the antigen concentration as well as patient sample antigen concentration for human interleukin 6 (IL-6) is generated through colorimetric analysis of IL-6 by ELISA. The resultant colorimetric results for the samples in FIG. 10 are captured and graphed in FIG. 11. The color obtained from the ELISA results are intense and highly contrasting, even to the naked eye. The standard curve obtained from ELISA experiments has been analyzed and plotted in FIG. 11 according to the absorbance obtained from colorimetric analysis, for higher color intensity corresponds to higher antigen concentration, and hence higher in absorbance value. The linearity and correlation of the antigen concentration to the color intensity of the chip have been extrapolated and proven statistically significant and powerful. The slope of the standard curve obtained is in the range of 0.5×10⁻⁵ to 1×10⁻⁵, and has been shown to be analytically sensitive to a difference of 10 pg/mL of antibody in the solution.

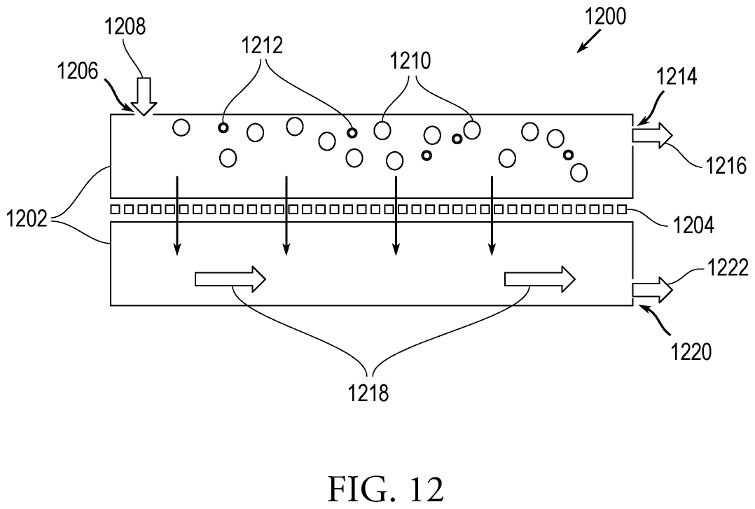

In another implementation, the cartridge includes a whole-blood filter, a substrate collection compartment, and an assay reagent-holding compartment. The whole-blood filter, integrated within the cartridge for performing subsequent immunoassays, employs a microfiltration design and transport mechanism. For example, the cartridge includes a whole-blood microfiltration mechanism 1200 as shown in FIG. 12. The whole-blood microfiltration mechanism 1200 includes a plurality of compartments 1202 with microchannels separated by a microfilter 1204. The mechanism 1200 includes an inlet 1206 of a top reservoir compartment of the plurality of compartments 1202. The inlet 1206 is configured to receive, for example, blood samples 1208 including blood cells 1210 and clotting factors 1212. The mechanism 1200 also includes an outlet 1214 configured to allow the outward flow 1216 of blood cells 1210. The microfilter 1204 facilitates the flow of plasma across a semipermeable membrane, allowing the separation of desired components from the blood sample. After filtration of the received blood samples 1208 by the microfilter 1204, serum 1218 is collected in a bottom filtrate compartment of the plurality of compartments 1202. The collected serum 1218 passes through an outlet 1220 configured to allow the outward flow 1222 of serum for external collection.

To construct the whole-blood microfiltration mechanism 1200 as shown in FIG. 12, in one implementation, an activated polycarbonate track etch (PCTE) membrane as the microfilter 1204 and layers of polydimethylsiloxane (PDMS) are bonded together to form a sandwich structure. The PCTE membrane with uniform pore size selectively allows the passage of plasma while retaining cellular components such as red blood cells and platelets. The separation of cellular components from plasma is achieved through the principle of size exclusion, in which the semipermeable membrane acts as a molecular sieve, permitting the passage of smaller molecules such as plasma while hindering the passage of larger cells.

As the blood sample is introduced into the top reservoir compartment, plasma is driven by hydrostatic pressure across the PCTE membrane into the filtrate microchannels below. The filtrate microchannels serve as conduits for the separated plasma, which are the directed into the first row of the assay reagent-holding compartment for the uptake into the chip for subsequent immunoassay analysis, while the blood cells outlet is directed to a substrate collection compartment. The assay reagent-holding compartment includes numerous rows of microwells alternately containing the reagents that need to be subsequently loaded into the chip and capillaric agent strips for unloading incubated reagents out of the chip, where the dimensions of the wells of the cartridges are complementary to the distance of the microchannels.

The apparatus 100 and process 400 have advantages and distinctions compared to conventional systems. The loading and unloading of substances into chips using capillary pressure instead of pumps greatly enhance the scalability of the apparatus 100. Each microchannel in a chip of a capillary-pressure-based systems acts as an individual capillary pump, hence a uniform and stable loading is achieved, which allows for better control and greater scalability, along with case of automation for the apparatus 100 and process 400.

In addition, the unloading of microchannels by the apparatus 100 and process 400 is advantageous compared to known paper or cotton-based unloading, in which the distinctions in the techniques include the aim and the versatility of agents transferrable through the substrate. For known paper or cotton-based unloading with general capillary action in lateral flow assays (LFA), or nitrocellulose PVDF, nylon membrane, or agarose gel used in blotting or gel electrophoresis, the purpose of a porous wick is to transfer molecules through the material, which is inefficient.

The apparatus 100 and process 400 facilitate the manipulation and transfer of fluids, in which molecules are bound and left within the microchannel. Furthermore, the substrate used in LFA, gel electrophoresis, or blotting is mostly applied to transferring biomolecules i.e. RNA, DNA, or proteins. For the unloading and emptying of microchannels, various agents such as cells, magnetic beads, etc. are manipulated, reloaded, or transferred through capillary action, increasing the possibilities of assay design.

Upon completion of the analysis, the apparatus 100 and process 400 provide a clear and concise readout 108, such as a display of the concentrations of the target biomarkers. Such information serves as a vital diagnostic tool for clinicians, enabling the clinicians to promptly assess the condition of a patient Based on the biomarker concentrations, healthcare professionals using the apparatus 100 and process 400 make informed decisions regarding appropriate medication and treatment strategies. The sample collection chip is conveniently disposable after each test, which eliminates the need for time-consuming and potentially error-prone cleaning and sterilization procedures between tests. The immediate disposal of the chip allows for seamless and efficient processing of subsequent samples, minimizing waiting times and expediting patient care.

Portions of the methods described herein can be performed by software or firmware in machine readable form on a tangible or non-transitory storage medium. For example, the software or firmware can be in the form of a computer program including computer program code adapted to cause the system to perform various actions described herein when the program is run on a computer or suitable hardware device, and where the computer program can be implemented on a computer readable medium. Examples of tangible storage media include computer storage devices having computer-readable media such as disks, thumb drives, flash memory, and the like, and do not include propagated signals. Propagated signals can be present in a tangible storage media. The software can be suitable for execution on a parallel processor or a serial processor such that various actions described herein can be carried out in any suitable order, or simultaneously.

It is to be further understood that like or similar numerals in the drawings represent like or similar elements through the several figures, and that not all components or steps described and illustrated with reference to the figures are required for all embodiments, implementations, or arrangements.

The terminology used herein is for the purpose of describing particular implementations only and is not intended to be limiting of the invention. As used herein, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third) is for distinction and not counting. For example, the use of “third” does not imply there is a corresponding “first” or “second.” Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

While the disclosure has described several exemplary implementations, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to implementations of the disclosure without departing from the essential scope thereof. Therefore, it is intended that the invention not be limited to the particular implementations disclosed, or to the best mode contemplated for carrying out this invention, but that the invention will include all implementations falling within the scope of the appended claims.

The subject matter described above is provided by way of illustration only and should not be construed as limiting. Various modifications and changes can be made to the subject matter described herein without following the example embodiments, implementations, and applications illustrated and described, and without departing from the true spirit and scope of the invention encompassed by the present disclosure, which is defined by the set of recitations in the following claims and by structures and functions or steps which are equivalent to these recitations.

What is claimed is:

1. A process, comprising:
retaining a sample in a microchannel of a chip;
retaining a predetermined substance in a well of a cartridge;
dipping the chip into the predetermined substance in the well;
guiding the predetermined substance into the microchannel by capillary action, thereby mixing the predetermined substance with the sample;
performing an analysis of the mixed sample;
generating an analysis message corresponding to the sample; and
outputting the analysis message.

2. The process of claim 1, further comprising, prior to retaining the sample, coating an interior surface of the microchannel with the sample.

3. The process of claim 1, further comprising, after the guiding, incubating the chip with the mixed sample.

4. The process of claim 1, further comprising, after the guiding, unloading the chip retaining the mixed sample.

5. The process of claim 4, wherein the unloading includes:
dipping the chip onto a porous wick or fiber pad.

6. The process of claim 1, further comprising, after the guiding, applying a signal generating substance to the sample.

7. The process of claim 1, wherein performing the analysis includes detecting a characteristic of the sample, and
wherein the analysis message includes the characteristic.

8. The process of claim 1, further comprising applying a magnetic field to the chip to perform magnetic bead fixation; and
wherein the sample includes a conjugated magnetic bead.

9. The process of claim 8, further comprising, after the magnetic bead fixation, removing the magnetic field to resuspend the magnetic beads in the mixed sample.

10. The process of claim 1, wherein outputting the analysis message includes displaying the analysis message on a display monitor.

11. A process, comprising:
retaining a sample in a microchannel of a chip;
mounting the chip on a movable member configured to move horizontally or vertically;
retaining a predetermined substance in a well of a cartridge;
positioning the cartridge on a base under the chip;
moving the chip downward to dip the chip into the predetermined substance in the well;
guiding the predetermined substance into the microchannel by capillary action, thereby mixing the predetermined substance with the sample;
moving the chip upward to remove the chip from the predetermined substance in the well;
moving the chip downward to dip the chip on a porous wick or fiber pad to unload the predetermined substance from the microchannel;
performing an analysis of the mixed sample;
generating an analysis message corresponding to the sample; and
outputting the analysis message.

12. The process of claim 11, wherein the movable member is a horizontally movable actuator.

13. The process of claim 11, wherein the movable member is a horizontally moving conveyor belt.

14. The process of claim 13, wherein the movable member is a vertical moving member.

15. The process of claim 14, wherein the vertical moving member is coupled to the horizontally movable conveyor belt.

16. The process of claim 14, wherein the vertical moving member is a vertically movable actuator.

17. The process of claim 11, further comprising, after the guiding, incubating the chip with the mixed sample.

18. The process of claim 11, wherein performing the analysis includes detecting a characteristic of the sample, and
wherein the analysis message includes the characteristic.

19. An apparatus, comprising:
a sequential loading and unloading unit including:
a movable member configured to retain a chip having a microchannel including a sample, and configured to move the chip at least vertically;
a base upon which a cartridge is disposed, wherein the cartridge includes a well retaining a predetermined substance;
wherein the movable member is configured to move the chip downward to dip the chip into the predetermined substance in the well to guide the predetermined substance into the microchannel by capillary action, thereby mixing the predetermined substance with the sample,
wherein the movable member is configured to move the chip upward to remove the chip from the predetermined substance in the well, and
wherein the movable member is configured to move the chip downward to dip the chip onto a porous wick or fiber pad to unload the predetermined substance from the microchannel;
a signal detection unit configured, responsive to the chip positioned therein, to perform an analysis of the mixed sample and to generate an analysis message corresponding to the sample; and
an output device configured to output the analysis message.

20. The apparatus of claim 19, wherein the analysis includes a detected characteristic of the sample, and
wherein the analysis message includes the detected characteristic.

21. The apparatus of claim 19, further comprising:
a whole-blood filter including a substrate collection compartment, and an assay reagent-holding compartment, to allow whole blood sample to be processed.

22. The apparatus of claim 19, further comprising:
an incubator configured to incubate the mixed sample.
